(19) United States
(12) Patent Application Publication
(10) Pub. No.: US 2023/0069297 A1
(43) Pub. Date: Mar. 2, 2023

(54) CONFORMABLE EARTIP WITH INTEGRATED MICROPHONE

(71) Applicant: Team IP Holdings, LLC, Fort Worth, TX (US)

(72) Inventor: Steve Iseberg, Crystal Lake, IL (US)

(21) Appl. No.: 17/896,665

(22) Filed: Aug. 26, 2022

Related U.S. Application Data
(60) Provisional application No. 63/239,075, filed on Aug. 31, 2021.

(51) Int. Cl. A61B 5/12; H04R 1/10; H04R 1/08; H04R 19/04; A61B 5/00

(52) U.S. Cl. CPC A61B 5/125; H04R 1/1016; H04R 1/08; H04R 19/04; H04R 1/1041; H04R 1/1075; A61B 5/6817; H04R 2201/003; A61B 2562/0204; A61B 2562/028

(57) ABSTRACT

Certain embodiments provide a hearing test probe apparatus including a hearing test probe, an eartip, and a microphone. The probe includes a speaker disposed within a probe body, and a mounting stem extending from the probe body and including a speaker sound channel. The eartip is detachably coupled to the mounting stem, and includes an eartip body having an ear insertion end and a probe insertion end opposite the ear insertion end. The eartip body defines a central opening extending from the ear insertion end to the probe insertion end. The central opening includes an eartip sound channel at the ear insertion end and an eartip mounting portion at the probe insertion end. The eartip mounting portion is configured to receive and hold the mounting stem of the probe within the central opening. The microphone is disposed within the eartip when the eartip is detachably coupled to the mounting stem.

### CROSS-REFERENCE TO RELATED APPLICATIONS/INCORPORATION BY REFERENCE

The present application claims priority under 35 U.S.C. § 119(e) to provisional application Ser. No. 63/239,075 filed on Aug. 31, 2021, entitled “CONFORMABLE EARTIP WITH INTEGRATED MICROPHONE.” The above referenced provisional application is hereby incorporated herein by reference in its entirety.

U.S. Pat. No. 9,432,760 issued to Iseberg, et al. on Aug. 30, 2016, is incorporated by reference herein in its entirety.

U.S. Pat. No. 9,197,956 issued to Iseberg, et al. on Nov. 24, 2015, is incorporated by reference herein in its entirety.

### FIELD

The present disclosure relates to hearing test probes. More specifically, the present disclosure relates to adapting a hearing test probe to an ear canal of a subject with a conformable eartip having a microphone disposed therein.

### BACKGROUND

Current hearing test probes commonly use industry-standard elastomeric ear-coupling cushions (i.e., eartips), which are available in a range of diameters intended to provide adequate coupling to a large range of ear canal sizes. Such eartips provide an acoustic pathway for the introduction of acoustic stimulus into the ear canal and the reception of acoustic responses from the ear canal. The acoustic pathway through these eartips is commonly of a diameter large enough to provide a low-impedance connection to the ear canal, providing for a smoother frequency response for both the acoustic transducer and the microphone as well as a reduced noise floor for the microphone. Although these industry-standard eartips have been successfully in use for decades and are well accepted by the audiology community, they are sometimes difficult to adapt to the subject's ear canal and are often uncomfortable for the subject. The limitation of the acoustic pathway dimension is directly in conflict with the desire to make the eartip comfortable for the subject, reducing the range of conformance possible in the eartip design.

Conformable eartips, which are both comfortable for the user and achieve quick acoustic seals to a range of ear canal sizes and shapes, have been available for other applications, such as earphones, hearing aids, and earplugs. Conforming eartips of this type are shown, for example, in the U.S. Pat. No. 9,432,760 by Iseberg, et al. and 9,197,956 by Iseberg, et al., both of which are incorporated by reference herein in their entirety.

Probes for use with hearing-measurement systems are subject to contamination from the cerumen or other biological material often found in human ear canals. Accordingly, eartips commonly used for hearing testing are designed to be replaceable, and either disposable, washable, or able to be disinfected. Also, the physical structure (commonly referred to as a probe tip or probe tube) comprising the acoustic pathway between the transducers and the eartip is often designed to be either cleanable or replaceable, providing a buffer between the sensitive probe components and the contamination. For example, FIG. 1 illustrates an exploded view of an exemplary replaceable probe tip 30 of a hearing test probe 10 as is known in the art. FIG. 2 illustrates an exploded side view of an exemplary hearing test probe 10 having a replaceable probe tube 30 as is known in the art. FIG. 3 illustrates a cross-sectional view of an exemplary hearing test probe 10 as is known in the art. Referring to FIG. 1, the hearing test probe 10 includes a probe body 40, a replaceable probe tip 30, and an eartip 20. Referring to FIGS. 2-3, the hearing test probe 10 includes an eartip 20, probe tube 30, probe body 40, end cap 45, microphone 50, microphone tube 55, drivers 60, stimulus channels 65, sealing and mating surface 70, and grommet 80. The replaceable probe tip 30 of FIG. 1 and the probe tube 30 of FIGS. 2 and 3 are examples of cleanable or replaceable acoustic interfaces as known in the art. While these designs provide for a more robust and reliable probe that can withstand the clinical environment, they necessarily extend the distance between the ear canal and the acoustic transducers 50.

Microphone noise floor is commonly an important specification for hearing test probes because it directly impacts the signal-noise ratio of the measured signals. The noise floor of common electret or micro-electromechanical systems (MEMs) microphones is lowest with an unrestricted acoustic port. The noise floor of the microphone increases as the acoustic impedance of the extended acoustic port is increased. The design of the acoustic path for the microphone indicates that the shortest length and the largest diameter or effective cross-sectional area causes the lowest increase in the nominal noise floor of the microphone transducer. Accordingly, it is advantageous to have the shortest acoustic path with the largest possible effective cross-sectional area to maintain the lowest possible noise floor.

Electret microphones have been made available in ever reducing sizes since their invention by Sessler and West in 1962. The introduction of MEMs microphones have allowed for further reduction in available microphone sizes as well as a significant reduction in cost largely due to widespread use in cellular phones.

Further limitations and disadvantages of conventional and traditional approaches will become apparent to one of skill in the art, through comparison of such systems with some aspects of the present disclosure as set forth in the remainder of the present application.

### SUMMARY

Certain embodiments of the present technology provide a conformable eartip having a microphone disposed therein, substantially as shown in and/or described in connection with at least one of the figures.

These and other advantages, aspects and novel features of the present disclosure, as well as details of an illustrated embodiment thereof, will be more fully understood from the following description and drawings.

### BRIEF DESCRIPTION OF THE DRAWINGS

FIG. 1 is an exploded view of an exemplary replaceable probe tip of a hearing test probe as is known in the art.

FIG. 2 is an exploded side view of an exemplary hearing test probe having a replaceable probe tube as is known in the art.

FIG. 3 is a cross-sectional view of an exemplary hearing test probe as is known in the art.

FIG. 4 is a cross-sectional view of an exemplary hearing test probe having a microphone disposed within an attached conformable eartip, in accordance with a first embodiment of the present technology.

FIG. 5 is a cut-away, exploded side perspective view of the exemplary hearing test probe detached from the conformable eartip, in accordance with the first embodiment of the present technology.

FIG. 6 is perspective view of the exemplary hearing test probe detached from the conformable eartip, in accordance with the first embodiment of the present technology.

FIG. 7 is perspective view of the exemplary hearing test probe detachably coupled to the conformable eartip, in accordance with the first embodiment of the present technology.

FIG. 8 is a side perspective view of an exemplary hearing test probe detached from a conformable eartip having a microphone embedded therein, in accordance with a second embodiment of the present technology.

FIG. 9 is a top, side perspective view of the exemplary hearing test probe detached from the conformable eartip having the microphone embedded therein, in accordance with the second embodiment of the present technology.

FIG. 10 is front, side perspective view of the exemplary hearing test probe detached from the conformable eartip having the microphone embedded therein, in accordance with the second embodiment of the present technology.

FIG. 11 is a cut-away, exploded side elevation view of the exemplary hearing test probe detached from the conformable eartip having the microphone embedded therein, in accordance with the second embodiment of the present technology.

FIG. 12 is a cut-away, exploded side perspective view of the exemplary hearing test probe detached from the conformable eartip having the microphone embedded therein, in accordance with the second embodiment of the present technology.

### DETAILED DESCRIPTION

Embodiments of the present technology provide a conformable eartip having a microphone disposed therein. In a representative embodiment, the hearing test probe allows for the use of eartips that easily and comfortably conform to a full range of sizes and varying shapes of human ear canals to provide an acoustic and pressure seal to the ear canal. Aspects of the present disclosure provide the technical effect of significantly reducing the length of the microphone acoustic path to maintain a low microphone noise floor, while allowing for the use of a comfortable and easily sealing conformable eartip by placing a MEMs microphone directly into the end of the mounting stem of the eartip. Various embodiments provide the technical effect of a significantly shorter acoustic path that allows for a smaller effective diameter without causing significant increase in the microphone noise floor.

Certain embodiments provide the technical effect of positioning the microphone close to the ear canal by providing a disposable eartip with the microphone embedded therein. Aspects of the present disclosure provide the technical effect of a shorter acoustic path providing a lower microphone noise floor and the ability to replace an entire eartip assembly in one component, ensuring a more sanitary use and eliminating eartip interface cleaning. Various embodiments provide the technical effect of allowing eartip assembly removal without having to touch a potentially contaminated component by a mechanical push button to release latches holding the eartip physically to the probe body.

The foregoing summary, as well as the following detailed description of certain embodiments will be better understood when read in conjunction with the appended drawings. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present technology is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

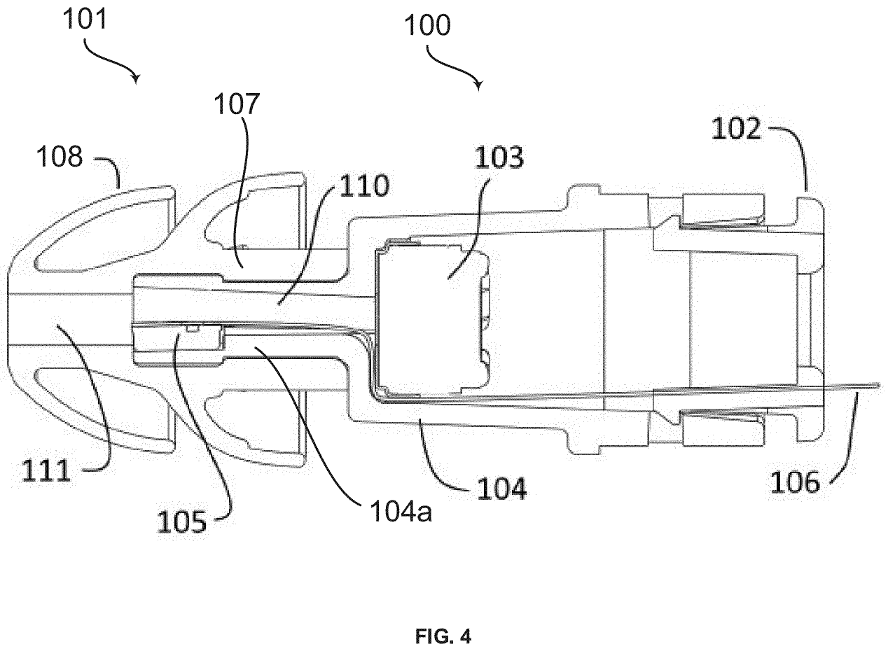

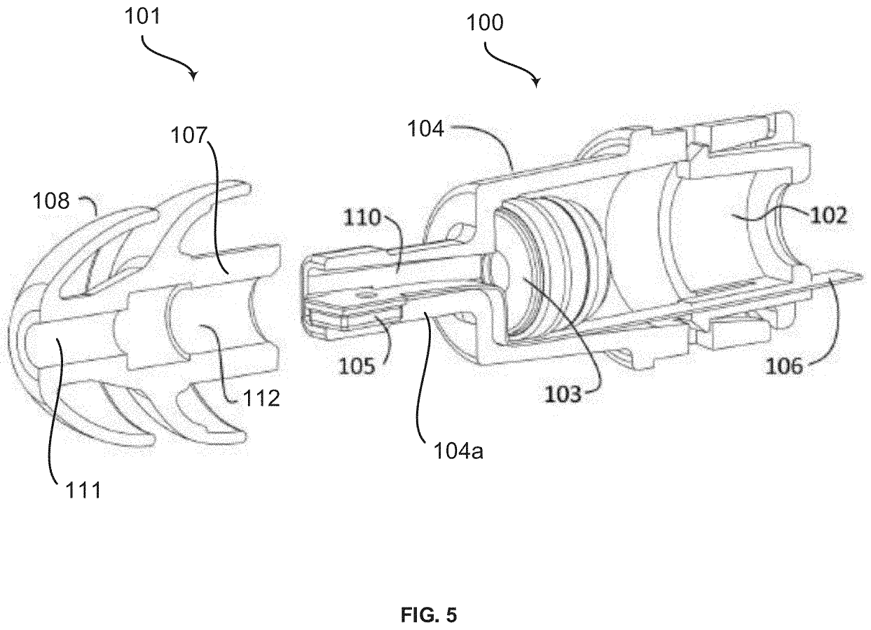

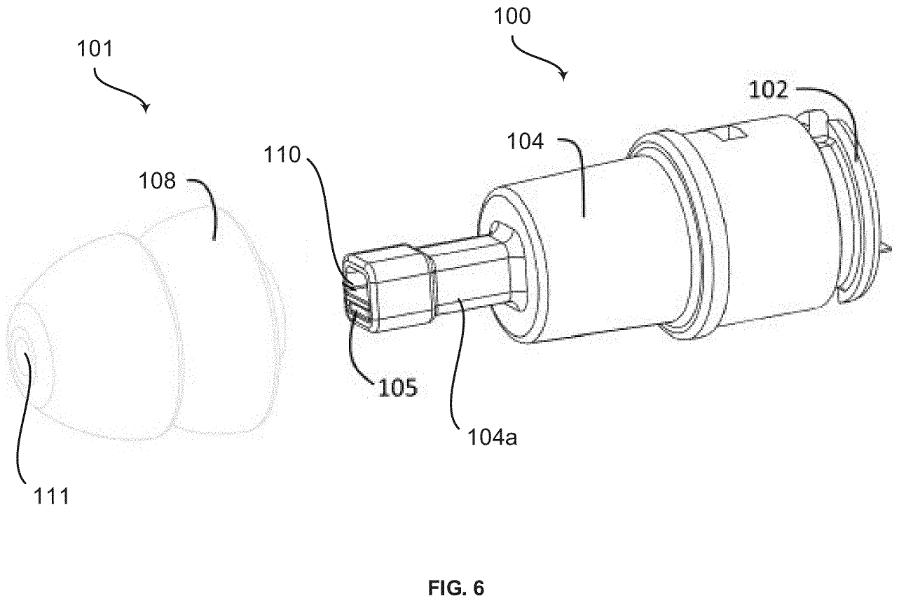

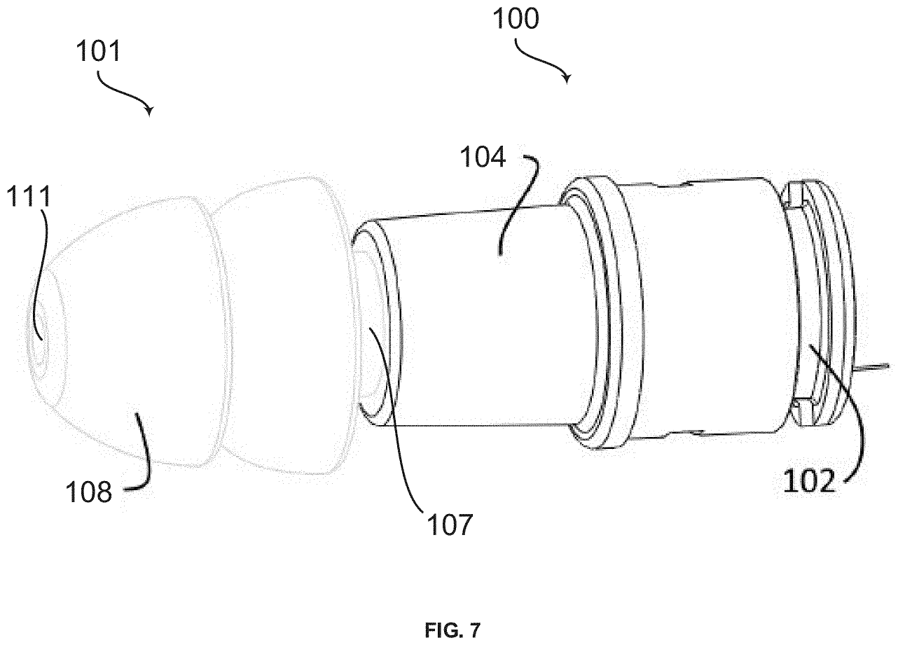

FIG. 4 is a cross-sectional view of an exemplary hearing test probe 100 having a microphone 105 disposed within an attached conformable eartip 101, in accordance with a first embodiment of the present technology. FIG. 5 is a cut-away, exploded side perspective view of the exemplary hearing test probe 100 detached from the conformable eartip 101, in accordance with the first embodiment of the present technology. FIG. 6 is perspective view of the exemplary hearing test probe 100 detached from the conformable eartip 101, in accordance with the first embodiment of the present technology. FIG. 7 is perspective view of the exemplary hearing test probe 100 detachably coupled to the conformable eartip 101, in accordance with the first embodiment of the present technology.

Referring to FIGS. 4-7, the hearing test probe 100 comprises a probe body 104, a probe cap 102, a speaker 103, a microphone 105, a flex connection 106, and a speaker sound channel 110, among other things. The probe cap 102 attaches to the probe body 104 and may be configured to secure a cable (not shown) for communicating electrical test signals to the flex connection 106 of the hearing test probe 100 and receiving the electrical response signals from the flex connection 106 of the probe 100. The probe cap 102 and probe body 104 may house various components of the hearing test probe 100, such as the speaker 103 and flex connection 106, among other things. In various embodiments, the probe cap 102 and probe body 104 may be a single, integrated component. The flex connection 106 electrically couples the cable to one or both of the speaker 103 and microphone 105. The probe body 104 comprises a probe body mounting stem 104a configured to be inserted into a conformable eartip 101. The probe body mounting stem 104a may comprise a speaker sound channel 110 extending from the speaker 103 disposed in the probe body 104 to a distal end of the probe body mounting stem 104a. The speaker 103 may be one or more moving coil drivers, balanced armature drivers, and/or any suitable sound transducer(s) that generate acoustic test stimulus from the electrical test signals received via the cable and flex connection 106, and transmit the acoustic test stimulus through the speaker sound channel 110 into the eartip 101 for output into an ear canal. The microphone 105 may be one or more MEMs microphones positioned directly in the distal end of the mounting stem 104a such that the microphone 105 is disposed within the conformable eartip 101 when the eartip 101 is detachably coupled to the mounting stem 104a.

The conformable eartip 101 may be made of an elastomer material, such as silicone, or any suitable material. The conformable eartip 201 may include an ear insertion end and a probe insertion end opposite the ear insertion end. The conformable eartip 201 may include an eartip body 107 and one or more flanges 108 extending from the eartip body 107 at an angle away from the eartip body 107 and toward the probe insertion end. The eartip body 107 defines a central opening 111, 112 extending from the ear insertion end to the probe insertion end. The central opening comprises an eartip sound channel 111 at the ear insertion end and an eartip mounting portion 112 at the probe insertion end. The eartip mounting portion 112 is configured to receive and hold the probe body mounting stem 104a of the probe body 104 within the central opening of the conformable eartip 101. The eartip sound channel 111 is configured to receive and carry acoustic responses from the ear canal at the ear insertion end to the microphone 105 disposed within the conformable eartip 101 in the distal end of the mounting stem 104a. The eartip sound channel 111 is configured to receive and carry acoustic test stimulus output from the speaker sound channel 110 at the distal end of the mounting stem 104a to an ear canal at the ear insertion end of the conformable eartip 101.

The conformable eartip 101 is detachably coupleable to the hearing test probe 100. The MEMs microphone 105 positioned directly into the end of the mounting stem 104a of the probe body 104 results in the microphone 105 being disposed within the eartip 101 when the eartip 101 is detachably coupled to the hearing test probe 100. The microphone 105 disposed within the eartip 101 significantly reduces the length of the microphone acoustic path defined by the eartip sound channel 111 to maintain a low microphone noise floor, while allowing for the use of a more comfortable and easily-sealing conformable eartip 101.

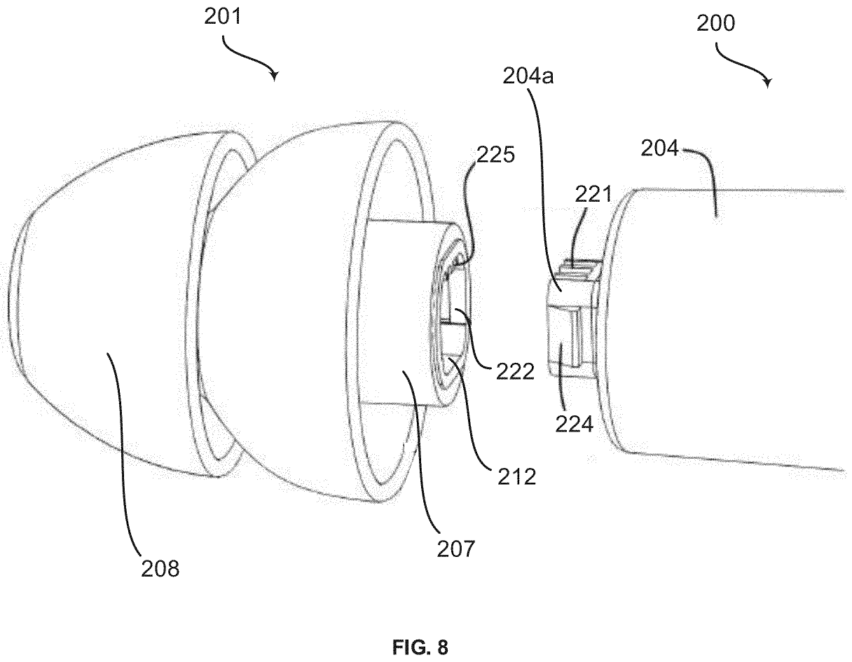

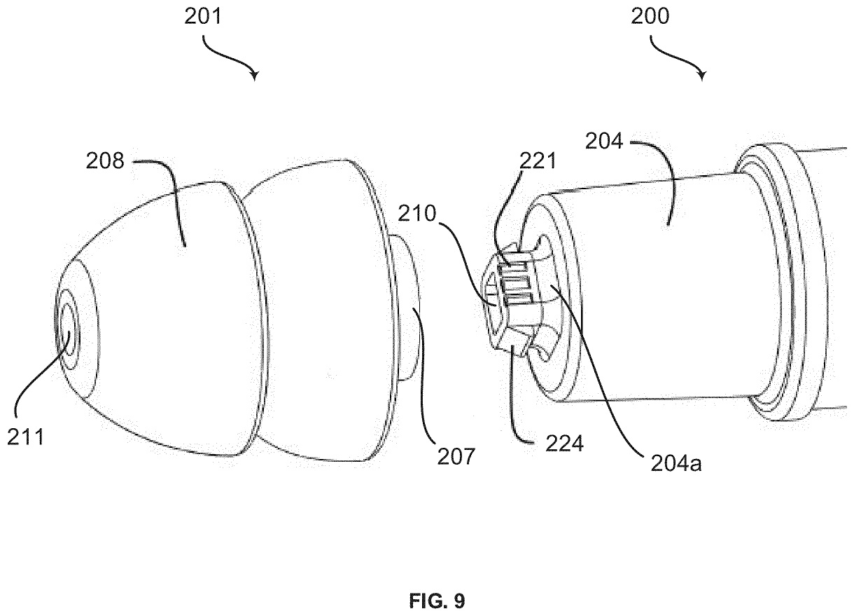

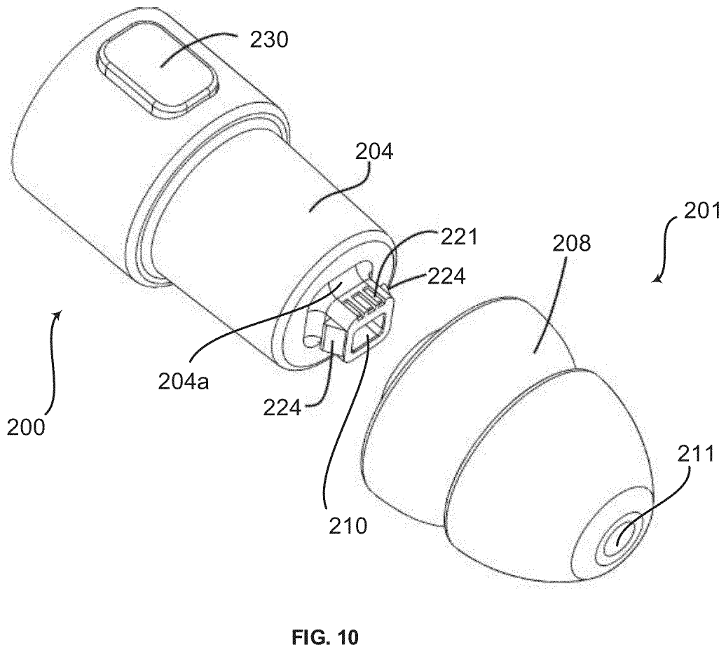

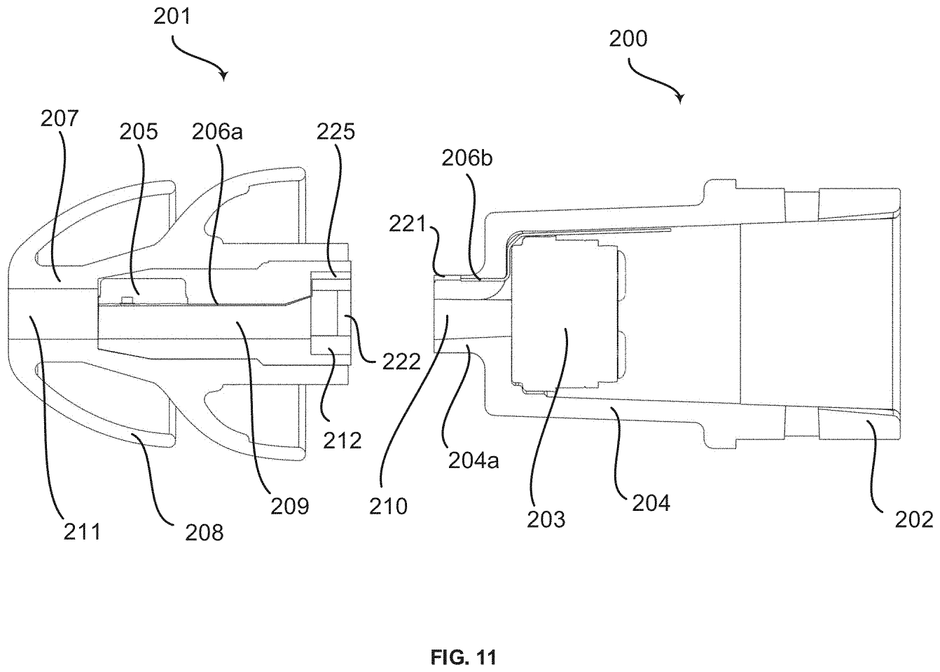

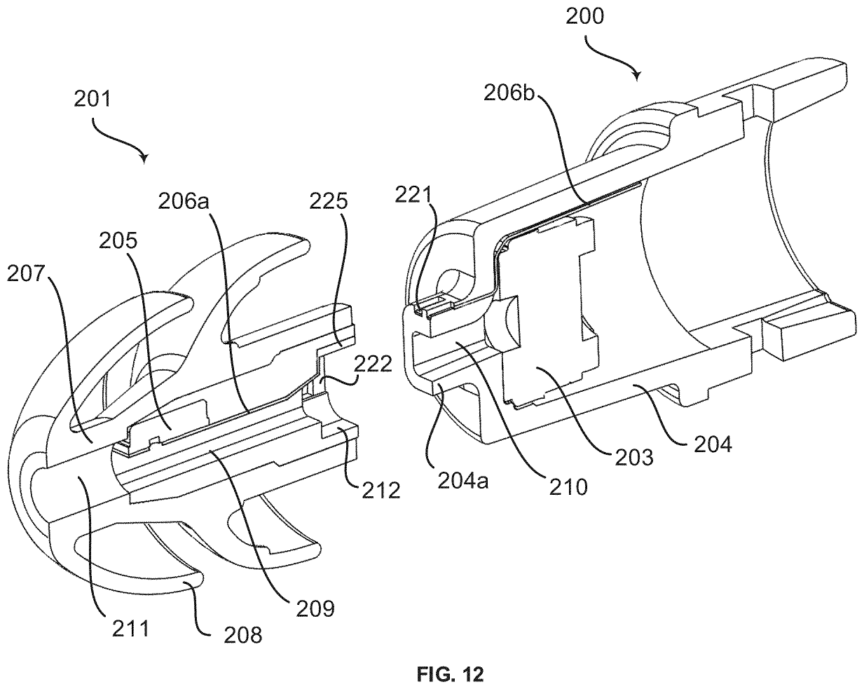

FIG. 8 is a side perspective view of an exemplary hearing test probe 200 detached from a conformable eartip 201 having a microphone 205 embedded therein, in accordance with a second embodiment of the present technology. FIG. 9 is a top, side perspective view of the exemplary hearing test probe 200 detached from the conformable eartip 201 having the microphone 205 embedded therein, in accordance with the second embodiment of the present technology. FIG. 10 is front, side perspective view of the exemplary hearing test probe 200 detached from the conformable eartip 201 having the microphone 205 embedded therein, in accordance with the second embodiment of the present technology. FIG. 11 is a cut-away, exploded side elevation view of the exemplary hearing test probe 200 detached from the conformable eartip 201 having the microphone 205 embedded therein, in accordance with the second embodiment of the present technology. FIG. 12 is a cut-away, exploded side perspective view of the exemplary hearing test probe 200 detached from the conformable eartip 201 having the microphone 205 embedded therein, in accordance with the second embodiment of the present technology.

Referring to FIGS. 8-12, the hearing test probe 200 comprises a probe body 204, a probe cap 202, a speaker 203, a flex connection 206b, and a speaker sound channel 210, among other things. The probe cap 202 attaches to the probe body 204 and may be configured to secure a cable (not shown) for communicating electrical test signals to the flex connection 206b of the hearing test probe 200 and receiving the electrical response signals from the flex connection 206b of the probe 200. The probe cap 202 and probe body 204 may house various components of the hearing test probe 200, such as the speaker 203 and flex connection 206b, among other things. In various embodiments, the probe cap 202 and probe body 204 may be a single, integrated component. The flex connection 206b electrically couples the cable to one or both of the speaker 203 and probe contacts 221. The probe body 204 comprises a probe body mounting stem 204a configured to mate with an eartip mounting portion 212 of a conformable eartip 202.

The probe body mounting stem 204a may comprise an acoustic connection 210, a mechanical connection 224, and an electrical connection 221 for detachably coupling to an acoustic connection 209, a mechanical connection 222, and an electrical connection 225 of the eartip mounting portion 212 of the conformable eartip 201. The acoustic connection 210 of the probe body mounting stem 204a is a speaker sound channel 210 extending from the speaker 203 disposed in the probe body 204 to a distal end of the probe body mounting stem 204a. The speaker sound channel 210 in the mounting stem 204a connects to a speaker sound channel 209 in the conformable eartip 201 when the eartip mounting portion 212 of the conformable eartip 201 is detachably coupled to the mounting stem 204a of the probe body 204. The speaker 203 may be one or more moving coil drivers, balanced armature drivers, and/or any suitable sound transducer(s) that generate acoustic test stimulus from the electrical test signals received via the cable and flex connection 206b, and transmit the acoustic test stimulus through the speaker sound channel 210 into the speaker sound channel 209 of the conformable eartip 201 for output into an ear canal.

The mechanical connection 222 of the probe body mounting stem 204a may comprise a locking mechanism 224 configured to detachably couple to an eartip body mating mechanism 222 of the eartip mounting portion 212 of the conformable eartip 201. For example, the eartip body mating mechanism 222 of the eartip mounting portion 212 of the conformable eartip 201 may comprise grooves, indentations, protrusions, or the like configured to latch onto the locking mechanism 224 of the probe body mounting stem 204a of the hearing test probe 200. The locking mechanism 224 may comprise latches that engage with the corresponding eartip body mating mechanism 222 of the conformable eartip 201 to securely hold the eartip mounting portion 212 on the probe body mounting stem 204a of the hearing test probe 200. The hearing test probe 200 may comprise a mechanical push button 230 configured to release the eartip 201 by swiveling the latches of the locking mechanism 224 inward to disengage the eartip body mating mechanism 222 from locking mechanism, thereby releasing the eartip mounting portion 212 of the eartip 201 from the mounting stem 204a of the hearing test probe 200. The release of the eartip mounting portion 212 of the eartip 201 from the mounting stem 204a of the hearing test probe 200 may eject the conformable eartip 201 from the hearing test probe 200 without a user having to touch the potentially contaminated component. Although the exemplary mechanical connection between the eartip mounting portion 212 of the conformable eartip 212 and the mounting stem 204a of the hearing test probe 200 is described above as pivotable latches of a locking mechanism 224 disengageably coupling to grooves, indentations, or protrusions of an eartip mounting portion 212, unless so claimed, the mechanical connection between the mounting stem 204a of the hearing test probe 200 and the eartip mounting portion 212 of the conformable eartip 212 is not so limited, and may include any suitable mechanical connection for detachably coupling the eartip mounting portion 212 of the conformable eartip 201 to the mounting stem 204a of the hearing test probe 200.

The electrical connection 221 of the probe body mounting stem 204a may comprise probe contacts 221 configured to detachably mate with microphone contacts 225 of the eartip mounting portion 212 of the conformable eartip 201. The probe contacts 221 of the probe body mounting stem 204a are electrically connected to the cable of the hearing test probe 200 via the flex connection 206b. The microphone contacts 225 of the eartip mounting portion 212 of the conformable eartip 201 are electrically connected to the microphone 205 embedded in the conformable eartip 201 via a flex connection 206a of the conformable eartip 201. The microphone 205 is configured to generate electrical response signals corresponding to acoustic responses received at the microphone 205. The electrical response signals are transmitted via the eartip flex connection 206a, microphone contacts 225, probe contacts 221, and probe flex connection 206b to the cable.

The conformable eartip 201 may be made of an elastomer material, such as silicone, or any suitable material. The conformable eartip 201 may include an ear insertion end and a probe insertion end opposite the ear insertion end. The conformable eartip 201 may include an eartip body 207 and one or more flanges 208 extending from the eartip body 207 at an angle away from the eartip body 207 and toward the probe insertion end. The eartip body 207 defines a central opening 209, 211, 212 extending from the ear insertion end to the probe insertion end. The central opening comprises an eartip sound channel 211 at the ear insertion end extending to an inlet of a microphone 205 embedded in the eartip 201, an eartip speaker sound channel 209 extending from the eartip sound channel 211 to the eartip mounting portion 212, and the eartip mounting portion 212 at the probe insertion end. The eartip mounting portion 212 is configured to receive and hold the probe body mounting stem 204a of the probe body 204 within the central opening of the conformable eartip 201. The eartip mounting portion 212 may comprise an acoustic connection 209, a mechanical connection 222, and an electrical connection 225 for detachably coupling to an acoustic connection 210, a mechanical connection 224, and an electrical connection 221 of the probe body mounting stem 204a of the hearing test probe 200 as described above. The eartip speaker sound channel 209 is configured to receive and carry acoustic test stimulus output from the speaker sound channel 210 at the distal end of the mounting stem 204a to the eartip sound channel 211. The eartip sound channel 211 is configured to receive and carry the acoustic test stimulus output from the eartip speaker sound channel 209 to an ear canal at the ear insertion end of the conformable eartip 201. The eartip sound channel 211 is configured to receive and carry acoustic responses from the ear canal at the ear insertion end to the microphone 205 embedded within the conformable eartip 201.

The conformable eartip 201 is detachably coupleable to the hearing test probe 200. The conformable eartip 201 comprises a microphone 205. The microphone 205 may be one or more MEMs microphones embedded within the conformable eartip 201 and having a microphone inlet exposed to the eartip sound channel 211 to receive acoustic responses from an ear canal of a wearer of the conformable eartip 201. In various embodiments, the eartip 201 having the embedded MEMs microphone, which typically costs less than 20% of similar electret microphone, is disposable. Accordingly, a representative embodiment provides a replaceable eartip 201 where the microphone 205 connects to the hearing test probe 200 via a series of contacts 221, 225 placed alongside the eartip retention mechanism 222, 224. The microphone 205 embedded in the eartip 201 provides a shorter acoustic path defined by the eartip sound channel 211 from the microphone 205 to the ear canal at the ear insertion end of the conformable eartip 201, providing a lower microphone noise floor. The ability to replace the entire eartip assembly 201 in one component ensures a more sanitary use and eliminates eartip interface cleaning. The mechanical push button 230 of the hearing test probe 200 configured to release the latches 224 holding the eartip 201 physically to the probe body 204 allows the eartip assembly 201 to be removed without having to touch the potentially contaminated component.

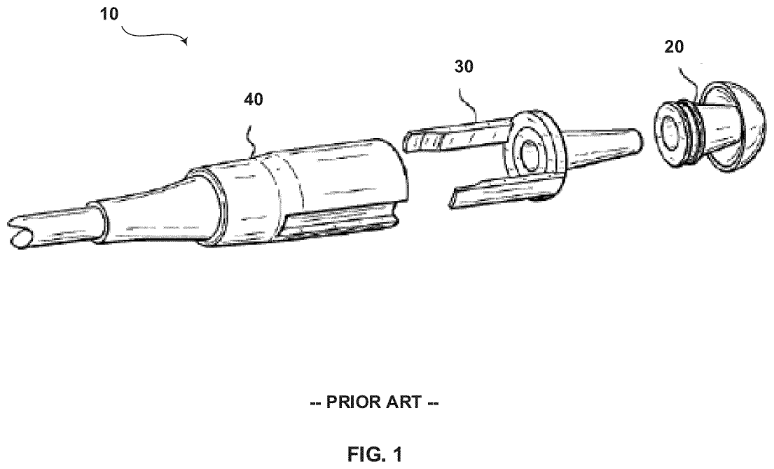

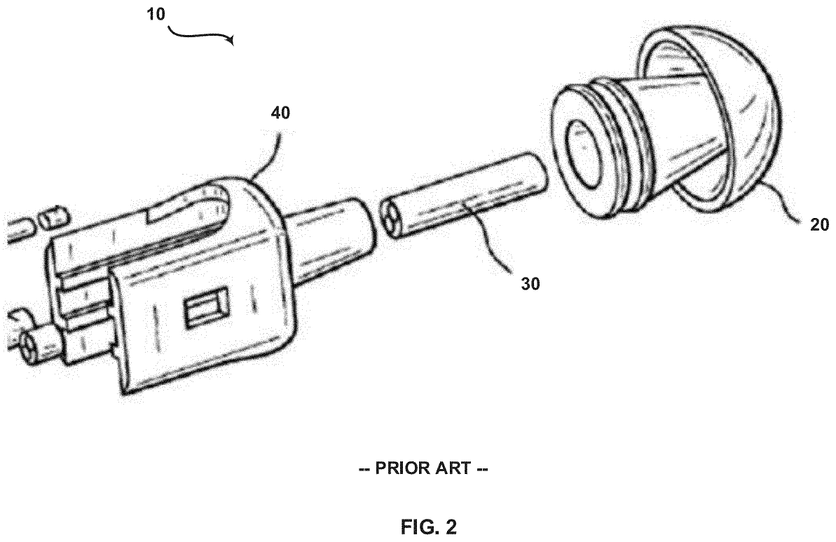

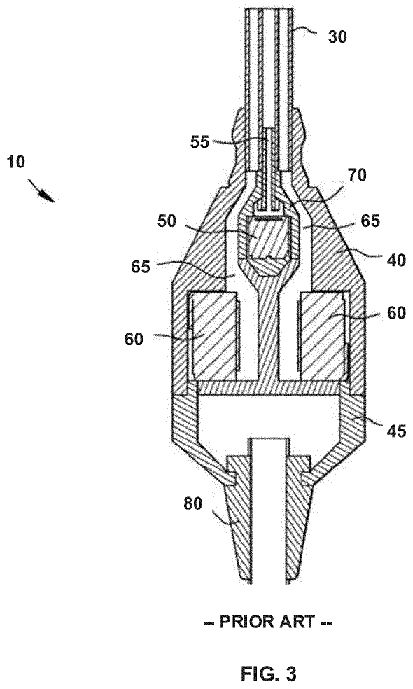

Referring to FIGS. 4-12, the microphone 105, 205 disposed within the eartip 101, 201 significantly reduces the length of the microphone acoustic path defined by the eartip sound channel 111, 211 to maintain a low microphone noise floor, while allowing for the use of a more comfortable and easily-sealing conformable eartip 101, 201. Specifically, instead of acoustic responses being carried an entire length of the eartip 20, entirely through a sound tube or replaceable probe tip 30, and into a probe housing 40 to microphone 50 as shown in FIGS. 1-3, the acoustic responses received via the conformable eartip 101, 201 of the disclosed embodiments are only carried from the ear insertion end through the eartip sound channel 111, 211 to an inlet of the microphone 105, 205 positioned at a central portion or front half of the conformable eartip 101, 201. The reduction in the length of the microphone acoustic path defined by the eartip sound channel 111, 211 allows for a smaller effective diameter of the eartip sound channel 111, 211 without causing a significant increase in the microphone noise floor. For example, prior art eartips 20 used with hearing test probes 10 typically include an interior sound channel having a diameter of at least 3 millimeters, while a diameter of the eartip sound channel 111, 211 of the disclosed embodiments is approximately 2 millimeters (i.e., 1.5-2.5 millimeters), which allows an outermost diameter of the conformable eartip 101, 201 to be smaller and more comfortable to a wearer.

Aspects of the present disclosure provide hearing test probes 100, 200 and eartips 101, 201 that allow the eartips 101, 201 to easily and comfortably conform to full range of sizes and varying shapes of human ear canals while providing an acoustic and pressure seal to the ear canal.

Various embodiments provide a hearing test probe apparatus comprising a hearing test probe 100, 200, an eartip 101, 201, and a microphone 105, 205. The hearing test probe 101, 201 comprises a probe body 104, 204, a speaker 103, 203, and a mounting stem 104a, 204a. The speaker 103, 203 is disposed within the probe body 104, 204 and is operable to generate acoustic test stimulus from received electrical test signals. The mounting stem 104a, 204a extends from the probe body 104, 204 and comprises a speaker sound channel 110, 210 operable to carry the generated acoustic test stimulus from the speaker 103, 203. The eartip 101, 201 is detachably coupled to the mounting stem 104a, 204a. The eartip 101, 201 comprises an eartip body 107, 207 having an ear insertion end and a probe insertion end opposite the ear insertion end. The eartip body 107, 207 defines a central opening 111, 112, 209, 211, 212 extending from the ear insertion end to the probe insertion end. The central opening 111, 112, 209, 211, 212 comprises an eartip sound channel 111, 211 at the ear insertion end and an eartip mounting portion 112, 212 at the probe insertion end. The eartip mounting portion 112, 212 is configured to receive and hold the mounting stem 104a, 204a of the hearing test probe 100, 200 within the central opening 111, 112, 209, 211, 212. The microphone 105, 205 is disposed within the eartip 101, 201 when the eartip 101, 201 is detachably coupled to the mounting stem 104a, 204a. The microphone 105, 205 is operable to receive an acoustic response via the eartip sound channel 111, 211.

In an exemplary embodiment, the microphone 105 is positioned directly in a distal end of the mounting stem 104a. In a representative embodiment, the microphone 105, 205 is one or more MEMs microphones. In various embodiments, the eartip 101, 201 comprises an elastomer material. In certain embodiments, the eartip 101, 201 comprises one or more flanges 108, 208 extending from the eartip body 107, 207 at an angle away from the eartip body 107, 207 and toward the probe insertion end. In an exemplary embodiment, the eartip sound channel 111, 211 is configured to receive the generated acoustic test stimulus from the speaker sound channel 110, 210 for output at the ear insertion end. In a representative embodiment, an entire length of a microphone acoustic path is defined by the eartip sound channel 111, 211. In various embodiments, the eartip sound channel 111, 211 is approximately two (2) millimeters in diameter. In certain embodiments, the microphone 105, 205 is positioned in a front half of the eartip 101, 201 toward the ear insertion end when the eartip 101, 201 is detachably coupled to the mounting stem 104a, 204a.

In a representative embodiment, the eartip 201 comprises the microphone 205 embedded in a front half of the eartip 201 toward the ear insertion end. In an exemplary embodiment, the eartip 201 comprises the microphone 205. In various embodiments, the eartip mounting portion 212 comprises an eartip acoustic connection 209 detachably coupled to a probe acoustic connection 210 of the mounting stem 204a, an eartip mechanical connection 222 detachably coupled to a probe mechanical connection 224 of the mounting stem 204a, and an eartip electrical connection 225 detachably coupled to a probe electrical connection 221 of the mounting stem 204a. In certain embodiments, the eartip acoustic connection is an eartip speaker sound channel 209. The probe acoustic connection is the speaker sound channel 210. In an exemplary embodiment, the hearing test probe apparatus comprises an eartip flex connection 206a electrically coupling the microphone 205 to the eartip electrical connection 225. The eartip electrical connection is microphone contacts 225. The probe electrical connection is probe contacts 221. In a representative embodiment, the eartip mechanical connection is an eartip body mating mechanism 222. The probe mechanical connection is a locking mechanism 224. In certain embodiments, the eartip body mating mechanism 222 comprises a groove, an indentation, and/or a protrusion. The locking mechanism comprises a latch 224 operable to engage with the groove, the indentation, and/or the protrusion of the eartip body mating mechanism 222. In various embodiments, the hearing test probe 200 comprises a mechanical push button 230 operable to swivel the latch 224 inward to release the eartip 201 from the hearing test probe 200 by disengaging the latch 224 from the groove, the indentation, and/or the protrusion of the eartip body mating mechanism 222.

Certain embodiments provide an eartip 201 comprising an eartip body 207, a central opening 209, 211, 212, and a microphone 205. The eartip body 207 comprises an ear insertion end and a probe insertion end opposite the ear insertion end. The central opening 209, 211, 212 extends through the eartip body 207 from the ear insertion end to the probe insertion end. The central opening 209, 211, 212 comprises an eartip sound channel 211 at the ear insertion end and an eartip mounting portion 212 at the probe insertion end. The eartip mounting portion 212 is configured to detachably couple to a hearing test probe 200. The microphone 205 is embedded within the eartip 201 and comprises a microphone inlet exposed to the eartip sound channel 211. The microphone 205 is operable to receive an acoustic response via the eartip sound channel 211.

In various embodiments, the microphone 205 is one or more MEMs microphones embedded in a front half of the eartip 201 toward the ear insertion end. In an exemplary embodiment, the eartip mounting portion 212 comprises an eartip speaker sound channel 209, probe contacts 225 electrically coupled to the microphone 205 via a flex connection 206a, and an eartip body mating mechanism 222.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, a structural component is “operable” and/or “configured” to perform a function whenever the structural component is made to or designed to perform the function, regardless of whether the function is performed.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A hearing test probe apparatus comprising:
a hearing test probe comprising:
a probe body;
a speaker disposed within the probe body and operable to generate acoustic test stimulus from received electrical test signals; and
a mounting stem extending from the probe body and comprising a speaker sound channel operable to carry the generated acoustic test stimulus from the speaker;
an eartip detachably coupled to the mounting stem, wherein:
the eartip comprises an eartip body having an ear insertion end and a probe insertion end opposite the ear insertion end;
the eartip body defines a central opening extending from the ear insertion end to the probe insertion end;
the central opening comprises an eartip sound channel at the ear insertion end and an eartip mounting portion at the probe insertion end; and
the eartip mounting portion is configured to receive and hold the mounting stem of the hearing test probe within the central opening; and
a microphone disposed within the eartip when the eartip is detachably coupled to the mounting stem, the microphone operable to receive an acoustic response via the eartip sound channel.

2. The apparatus of claim 1, wherein the microphone is positioned directly in a distal end of the mounting stem.

3. The apparatus of claim 1, wherein the microphone is one or more micro-electromechanical systems (MEMs) microphones.

4. The apparatus of claim 1, wherein the eartip comprises an elastomer material.

5. The apparatus of claim 1, wherein the eartip comprises one or more flanges extending from the eartip body at an angle away from the eartip body and toward the probe insertion end.

6. The apparatus of claim 1, wherein the eartip sound channel is configured to receive the generated acoustic test stimulus from the speaker sound channel for output at the ear insertion end.

7. The apparatus of claim 1, wherein an entire length of a microphone acoustic path is defined by the eartip sound channel.

8. The apparatus of claim 1, wherein the eartip sound channel is approximately two (2) millimeters in diameter.

9. The apparatus of claim 1, wherein the microphone is positioned in a front half of the eartip toward the ear insertion end when the eartip is detachably coupled to the mounting stem.

10. The apparatus of claim 1, wherein the eartip comprises the microphone embedded in a front half of the eartip toward the ear insertion end.

11. The apparatus of claim 1, wherein the eartip comprises the microphone.

12. The apparatus of claim 11, wherein the eartip mounting portion comprises:
an eartip acoustic connection detachably coupled to a probe acoustic connection of the mounting stem;
an eartip mechanical connection detachably coupled to a probe mechanical connection of the mounting stem; and
an eartip electrical connection detachably coupled to a probe electrical connection of the mounting stem.

13. The apparatus of claim 12, wherein:
the eartip acoustic connection is an eartip speaker sound channel; and
the probe acoustic connection is the speaker sound channel.

14. The apparatus of claim 12, comprising an eartip flex connection electrically coupling the microphone to the eartip electrical connection, and wherein:
the eartip electrical connection is microphone contacts; and
the probe electrical connection is probe contacts.

15. The apparatus of claim 12, wherein:
the eartip mechanical connection is an eartip body mating mechanism; and
the probe mechanical connection is a locking mechanism.

16. The apparatus of claim 15, wherein:
the eartip body mating mechanism comprises a groove, an indentation, and/or a protrusion; and
the locking mechanism comprises a latch operable to engage with the groove, the indentation, and/or the protrusion of the eartip body mating mechanism.

17. The apparatus of claim 16, wherein the hearing test probe comprises a mechanical push button operable to swivel the latch inward to release the eartip from the hearing test probe by disengaging the latch from the groove, the indentation, and/or the protrusion of the eartip body mating mechanism.

18. An eartip comprising:
an eartip body having an ear insertion end and a probe insertion end opposite the ear insertion end;
a central opening extending through the eartip body from the ear insertion end to the probe insertion end, wherein:
the central opening comprises an eartip sound channel at the ear insertion end and an eartip mounting portion at the probe insertion end; and
the eartip mounting portion is configured to detachably couple to a hearing test probe; and
a microphone embedded within the eartip and comprising a microphone inlet exposed to the eartip sound channel, the microphone operable to receive an acoustic response via the eartip sound channel.

19. The eartip of claim 18, wherein the microphone is one or more micro-electromechanical systems (MEMs) microphones embedded in a front half of the eartip toward the ear insertion end.

20. The eartip of claim 18, wherein the eartip mounting portion comprises:
an eartip speaker sound channel;
probe contacts electrically coupled to the microphone via a flex connection; and
an eartip body mating mechanism.
